(19) United States
(12) Patent Application Publication
(10) Pub. No.: US 2024/0366527 A1
(43) Pub. Date: Nov. 7, 2024

(54) HONOKIOL LIPOSOME TRANSDERMAL GEL, PREPARATION METHOD AND USE THEREOF

(71) Applicant: CHENGDU JINRUI FOUNDATION BIOTECH CO., LTD., Chengdu (CN)

(72) Inventors: NENG QIU, Chengdu (CN); Fangui Kong, Chengdu (CN); Haoyu Ye, Chengdu (CN); Zhanyang Li, Chengdu (CN); Li Ding, Chengdu (CN)

(21) Appl. No.: 18/775,920

(22) Filed: Jul. 17, 2024

(30) Foreign Application Priority Data
Jul. 19, 2023 (CN) 2023108827685

(51) Int. Cl. A61K 31/05; A61K 9/06; A61K 9/127; A61K 47/10; A61P 17/06

(52) U.S. Cl. CPC A61K 31/05; A61K 9/06; A61K 9/1271; A61K 9/1277; A61K 47/10; A61P 17/06

(57) ABSTRACT

A honokiol liposome transdermal gel includes by weight percentage: 1-40% honokiol liposome and 10-85% gel matrix. The honokiol liposome comprises phospholipid, honokiol, cholesterol, and polyethylene glycol or pegylated phospholipid, in a weight ratio of 2-10:1:0.3-0.6:0.1-0.3; and the gel matrix is poloxamer. The invention achieves an encapsulation efficiency of honokiol greater than 90%, the honokiol liposome transdermal gel has high transdermal efficiency, rapid and sustained absorption, and high stability, and shows good therapeutic effects for skin diseases.

### RELATED APPLICATIONS

This application claims the benefit of priority to Chinese Patent Application No. 202310882768.5 filed on Jul. 19, 2023, which is hereby incorporated by reference in its entirety.

### FIELD OF THE INVENTION

The present invention relates to the technical field of liposome formulation preparation, particularly to a honokiol liposome transdermal gel, a preparation method and a use thereof.

### BACKGROUND OF THE INVENTION

Magnolia officinalis, the dried bark of the perennial Magnoliaceae plant Magnolia biloba, is a commonly used clinical traditional Chinese medicine. Its main active ingredients include magnolol, honokiol, isomagnolol, tetrahydromagnolol, and magnoflorine, with magnolol and honokiol having the highest content.

Studies have shown that honokiol possesses a wide range of pharmacological effects, including antibacterial, anti-inflammatory, anxiolytic, morphine withdrawal inhibition, catecholamine secretion inhibition, calmodulin antagonism, antiviral, antitumor, and anti-aging activities. Therefore, honokiol has significant clinical application prospects. In recent years, the potent antibacterial and anti-inflammatory effects of honokiol have attracted researchers' attention to its dermatological applications, especially for topical drugs in treating skin diseases, which have unparalleled advantages. However, the low water solubility and bioavailability of the honokiol hinder its transdermal absorption, which prevents it from penetrating deep into the skin, and significantly limits its clinical application in topical formulations.

Chinese patent application publication CN107115347A discloses a liposome antiallergic agent and cosmetic for repairing hormone-dependent dermatitis, including the following components by weight percentage: 8-15% hydrogenated lecithin, 1-10% cholesterol, 0.5-2% ceramide 3, 2-5% ubiquinone, 2-5% honokiol, 10-20% butanediol, 0.1-0.5% citric acid, 0.3-1.5% sodium citrate, and water as the remainder. The components of the antiallergic agent synergistically enhance each other's effects, thereby alleviating inflammation, promoting keratin renewal, and quickly repairing hormone-dependent dermatitis. However, this patent application does not conduct in-depth research on the performance of the liposome or the skin penetration effects, nor does it study the therapeutic effects of honokiol liposome on skin diseases.

Chinese patent application publication CN113440426A discloses a honokiol double-encapsulated formulation, where honokiol is encapsulated by lignin or its derivatives and liposome, including by weight percentage: 1-5% honokiol, 1-10% lecithin, 1-15% liquid oil, 1-15% emulsifier, 0.1-5% lignin or its derivatives, and deionized water as the remainder. The total penetration rate of the honokiol in the stratum corneum test reaches 98.4%. However, lignin is a complex polymer of coumaryl alcohol, coniferyl alcohol, and sinapyl alcohol, lacking a definite basic structure, exhibiting significant heterogeneity, which affects the dispersion, compatibility, and stability of the drug components in the polymer.

Current research and development of honokiol liposome transdermal formulations still have corresponding deficiencies. The functionality is often limited to skincare effects, lacking detailed and in-depth research on the performance and transdermal effects of the liposome. There are no external formulations for treating skin diseases on the market. Therefore, it's an urgent technical problem to how to provide a honokiol liposome transdermal formulation that achieves high transdermal absorption efficiency, good product stability, and aims to treat skin diseases.

The references are as follows:
1. ZHANG Mingfa, SHEN Yaqin. Research advances on anti-inflammation and their mechanisms of extract, magnolol and honokiol from Magnoliae officinalis Cortex [J]. Drug Evaluation Research, 2021;
2. LIN C F, HWANG T L, SUWAYEH S A A, et al. Maximizing dermal targeting and minimizing transdermal penetration by magnolol/honokiol methoxylation [J]. Int J Pharm, 2013.

### SUMMARY OF THE INVENTION

For solving the aforementioned technical problems, the present invention provides a honokiol liposome transdermal gel, a preparation method and a use thereof. Such a honokiol liposome transdermal gel aims to improve the efficiency of honokiol liposome transdermal absorption, enhance product stability, and clarify therapeutic effects.

To achieve the above objective, the technical solution of the invention is as follows.

The present invention provides a honokiol liposome transdermal gel, comprising by weight percentage: 1-40% honokiol liposome and 10-85% gel matrix; wherein the honokiol liposome comprises phospholipid, honokiol, cholesterol, and polyethylene glycol or pegylated phospholipid, in a weight ratio of 2-10:1:0.3-0.6:0.1-0.3; and the gel matrix is poloxamer.

As an embodiment, a weight ratio of the honokiol liposome to the poloxamer is 1:1-1:10, preferably 1:2-1:6.

As an embodiment, the poloxamer is selected from any one or a combination of poloxamer 407, poloxamer 188, poloxamer 338, poloxamer 237, poloxamer 124, poloxamer 181, poloxamer 182, and poloxamer 331. Preferably, the poloxamer is poloxamer 407 and/or poloxamer 188.

As an embodiment, the honokiol liposome transdermal gel further includes an additive selected from any one or a combination of antioxidant, transdermal enhancer, moisturizer, pH adjuster, preservative, and thickener.

The antioxidant, transdermal enhancer, moisturizer, pH adjuster, preservative, and thickener in the present invention are existing technologies and are conventional additives used in hydrogel formulations. Their usage amount is the conventional dosage in this field.

The antioxidant in the present invention can be any one or a combination of sodium sulfite, sodium bisulfite, sodium thiosulfate, sodium metabisulfite, ascorbic acid, ascorbyl palmitate, propyl gallate, tocopherol, butylated hydroxyanisole, butylated hydroxytoluene, and edetate disodium, which is not limited to these however.

The transdermal enhancer in the present invention can be any one or a combination of laurocapram, propylene glycol, ethanol, menthol, peppermint oil, methyl salicylate, borneol, oleic acid, oleate, eucalyptus oil, DMSO, azone, lauric acid, which is not limited to these however.

The moisturizer in the present invention can be any one or a combination of glycerin, propylene glycol, hyaluronic acid, 1,3-butanediol, 1,2-hexanediol, sorbitol, maltitol, which is not limited to these however.

The preservative in the present invention can be any one or a combination of ethanol, sorbic acid, potassium sorbate, benzoic acid, sodium benzoate, chlorobutanol, benzalkonium chloride, benzalkonium bromide, methylparaben, ethylparaben, propylparaben, cysteine, chlorhexidine hydrochloride, ethylparaben, which is not limited to these however.

The pH adjuster in the present invention can be any one or a combination of sodium bicarbonate, sodium hydroxide, potassium hydroxide, hydrochloric acid, phosphates, citric acid and its salts, glacial acetic acid, triethanolamine, which is not limited to these however.

The thickener in the present invention can be any one or a combination of hydroxypropyl methylcellulose, methylcellulose, sodium hyaluronate, polyvinyl alcohol, polycarbophil, chondroitin sulfate, polyvinylpyrrolidone, which is not limited to these however.

The polyethylene glycol or pegylated phospholipid in the present invention preferably has a molecular weight of 800-20000 Da, preferably 1000-8000 Da.

The polyethylene glycol in the present invention can be any one or a combination of polyethylene glycol 1000, polyethylene glycol 2000, polyethylene glycol 4000, and polyethylene glycol 8000, which is not limited to these however.

The pegylated phospholipid in the present invention are existing technologies, which are amphiphilic polymer molecules including polyethylene glycol molecules as the hydrophilic block and phospholipid molecules as the hydrophobic block, and formed by covalently bonding the polyethylene glycol molecules to the nitrogenous base on the phospholipid molecules. The pegylated phospholipid may be any one or a combination of phosphatidylcholine-polyethylene glycol (PC-PEG), phosphatidylethanolamine-polyethylene glycol (PE-PEG), distearoylphosphatidylethanolamine-polyethylene glycol (DSPE-PEG), distearoylphosphatidylcholine-polyethylene glycol (DSPC-PEG), dimyristoylphosphatidylethanolamine-polyethylene glycol (DMPE-PEG), dipalmitoylphosphatidylcholine-polyethylene glycol (DPPC-PEG), dipalmitoylphosphatidylethanolamine-polyethylene glycol (DPPE-PEG), hydrogenated soybean phosphatidylethanolamine-polyethylene glycol (HSPE-PEG), dioleoylphosphatidylethanolamine-polyethylene glycol (DOPE-PEG), diarachidoylphosphatidylethanolamine-polyethylene glycol (DEPE-PEG), distearoylphosphatidylethanolamine-methoxy polyethylene glycol (DSPE-MPEG), distearoylphosphatidylethanolamine-polyethylene glycol-amino (DSPE-PEG-NH2), distearoylphosphatidylethanolamine-polyethylene glycol-carboxyl (DSPE-PEG-COOH), which is not limited to these however.

The present invention further provides a preparation method of the honokiol liposome transdermal gel, which includes the following steps:
(1) preparation of honokiol liposome: taking and uniformly mixing phospholipid, cholesterol, polyethylene glycol or pegylated phospholipid, and honokiol, adding organic solvent to fully dissolve, evaporating to remove the organic solvent to obtain a uniform phospholipid film, adding water to the phospholipid film, stirring for 1-1.5 h, homogenizing to obtain a liposome solution; and filtering the liposome solution at 2-10° C. with a filter of 0.22 μm to obtain the honokiol liposome; and
(2) preparation of honokiol liposome transdermal gel: stirring poloxamer with water for 0.5-5 h until fully swollen, then adding the honokiol liposome obtained in step (1) and an additive during stirring, and stirring for 20-40 minutes to obtain the honokiol liposome transdermal gel.

As an embodiment, the organic solvent in step (1) is selected from any one or a combination of anhydrous ethanol, chloroform, methylene chloride, and methanol. Preferably, it is a mixture of chloroform and anhydrous ethanol in a volume ratio of 1:1-1:10, or a mixture of chloroform and methanol in a volume ratio of 1:1-1:10.

As an embodiment, the poloxamer in step (2) is stirred with water of 2-10° C., with a swelling temperature of 2-10° C., and a swelling time of 24-72 hours.

The present invention further provides a use of the honokiol liposome transdermal gel in a preparation of a formulation for preventing and/or treating psoriasis.

The advantages of the present invention are as follows.

The honokiol liposome transdermal gel provided by the present invention combines liposome technology with gel technology. The honokiol liposome is incorporated into the poloxamer matrix. By controlling the proportion of raw materials and auxiliary materials in the preparation process of the liposome, especially by controlling the proportion of honokiol liposome to the gel-matrix poloxamer, high transdermal absorption effect is achieved, therefore the honokiol liposome transdermal gel has high transdermal efficiency, rapid and sustained absorption, and high stability, and shows good therapeutic effects for skin diseases.

### BRIEF DESCRIPTION OF THE DRAWINGS

FIG. 1 shows the cumulative permeation per unit area of different formulations on normal skin in Example 2.

FIG. 2 shows the cumulative permeation per unit area of different formulations on model skin in Example 2.

FIG. 3 shows cumulative permeation per unit area of liposome transdermal gels with different matrixes on model skin in Example 3.

### DETAILED DESCRIPTION OF ILLUSTRATED EMBODIMENTS

To clarify the objectives, technical solutions, and advantages of the present invention, the following examples are provided for specific descriptions. It should be noted that these examples are only for explanation and illustration, and not for limiting the invention. Some non-essential improvements and adjustments made by those skilled in the art based on the disclosed invention still fall within the protection scope of the present invention.

The honokiol used in the specific embodiments of the present invention has a purity of ≥99%.

### Example 1: Preparation of Formulations

### Step 1—Preparation of Honokiol Liposome Solution

Phospholipid, honokiol (10 g), cholesterol, polyethylene glycol-2000 in a weight ratio of 3:1:0.4:0.2 were taken and mixed uniformly, and then fully dissolved in anhydrous ethanol, then evaporated at 40° C. and 120 rpm to remove the organic solvent to obtain a uniform phospholipid film. The phospholipid film was hydrated with water for injection at 50° C., and stirred magnetically at 2000 rpm for 1 hour, then the product was homogenized in a high-pressure homogenizer at a homogenization pressure of 15,000 psi, and then the solution was filtered through a filter of 0.22 μm at 4° C. to obtain the honokiol liposome solution.

Upon measurement, the prepared honokiol liposome had an average particle size of 118.2 nm, a PDI value of 0.219, a zeta potential of −33.2 mV, and an encapsulation efficiency of honokiol greater than 90%.

### Step 2—Preparation of Honokiol Liposome Cream

The cream base, by weight, includes 1% honokiol as the drug content and remaining components including 10% glyceryl monostearate, 5% glycerin, 2.5% petroleum jelly, 8% liquid paraffin, 2.5% triethanolamine, 5% lanolin, and water as the reminder. The glyceryl monostearate, lanolin, petroleum jelly, and liquid paraffin were heated in a water bath at 70° C. to obtain the oil phase; then the glycerin and triethanolamine were mixed and dissolved in water to obtain the aqueous phase, and heated in a water bath to 70° C.; and then the aqueous phase was slowly added to the oil phase under stirring, and kept stirring until room temperature; and the prepared honokiol liposome solution was slowly added and stirred evenly to obtain the honokiol liposome cream.

### Step 3—Preparation of Honokiol Liposome Transdermal Gel

The gel, by weight, includes 1% honokiol as the drug content and remaining components including 14% poloxamer 407 and water as the reminder. The poloxamer was added to water of 4° C. and stirred for 3 hours, and fully swollen at 4° C. for 24 hours; then the prepared honokiol liposome solution was added under stirring, and stirred for 30 minutes to obtain the honokiol liposome transdermal gel.

### Step 4-Preparation of Honokiol Micelle Gel

The gel, by weight, includes 1% honokiol as the drug content and the remaining components including 30% poloxamer 407, 4% propylene glycol, and water as the reminder. 15% poloxamer 407 and half of the prescription amount of water were dissolved in an ice bath at 4° C., honokiol was added under ultrasonic conditions, sonicated for 30 minutes, and filtered with a filter of 0.22 μm to obtain a suspension. The remaining poloxamer 407 and water were mixed and swollen at 4° C. for 24 hours, then propylene glycol was added to obtain solutions, and then the solutions were added to the suspension and stirred for 40 minutes to obtain the honokiol micelle gel.

### Example 2: In Vitro Transdermal Penetration Experiment

### Step 1—Permeation Experiment on Normal Skin

Healthy male mice were selected, with abdominal hair shaved. After euthanasia, the skin was excised, subcutaneous fat and connective tissue were removed, and the skin was washed with saline and cut into uniform square pieces for use.

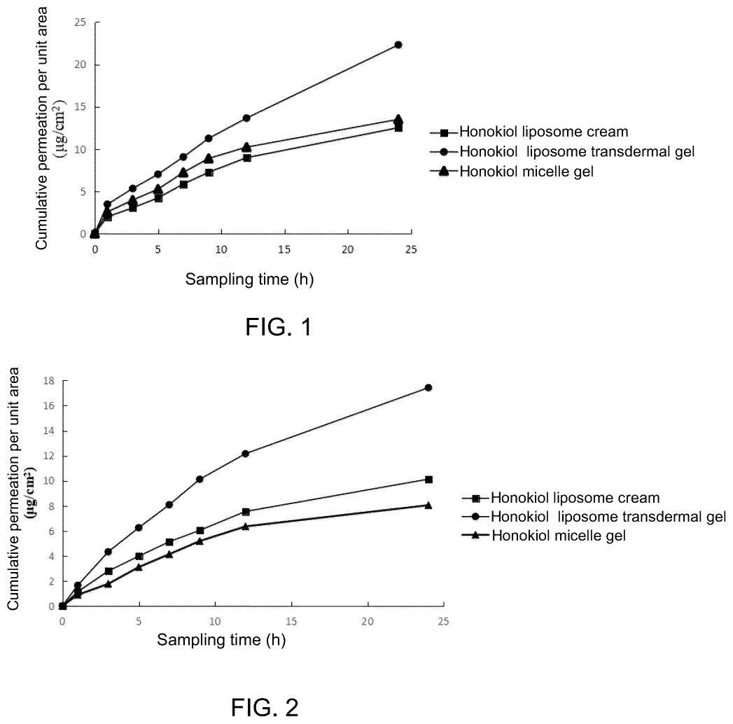

Using a Franz diffusion cell, the mice skin was fixed between the donor compartment and the receptor compartment, with the epidermis facing the donor compartment. A stir bar and saline were contained in the receptor compartment for removing air bubbles. Three sample formulations (honokiol liposome cream, honokiol liposome transdermal gel, and honokiol micelle gel) were respectively added to the donor compartment, maintaining the same drug content of honokiol in the samples. The system was kept at a constant temperature of 32° C. with magnetic stirring at 400 rpm. Samples of 1 ml were taken from the receptor compartment respectively at 1^{st}, 3^{rd}, 5^{th}, 7^{th}, 9^{th}, 12^{th}, and 24^{th} hour, with equal volumes of synthermal saline added back. The samples were respectively filtered through a filter of 0.45 μm and analyzed for measurement. The results for the drug content of honokiol and the cumulative permeation per unit area (Qn) are shown in FIG. 1.

As seen in FIG. 1, the cumulative permeation per unit area (Qn) over 0-24 hours was highest for the honokiol liposome transdermal gel, followed by the honokiol micelle gel, and then the honokiol liposome cream. The result shows small difference between the liposome cream and the honokiol micelle gel, but shows significant superiority in the liposome transdermal gel.

### Step 2—Permeation Experiment on Model Skin

Healthy mice were selected and housed individually, with backs shaved. Imiquimod cream (50 mg) was applied to their backs daily. After 5 days, the mice were euthanized, and psoriatic-like skin from their backs was excised. Subcutaneous fat and connective tissue were removed, and the skin was washed with saline and cut into uniform square pieces for use.

Using the same method as in Example 1, the cumulative permeation per unit area (Qn) of different formulations was measured on the model skin. Results are shown in FIG. 2.

As seen in FIG. 2, the cumulative permeation per unit area (Qn) over 0-24 hours was highest for the honokiol liposome transdermal gel, followed by the honokiol liposome cream, and then the honokiol micelle gel. The honokiol liposome transdermal gel had the highest cumulative permeation on psoriatic model skin.

### Example 3: Carrier Screening of Liposome Transdermal Gel

Step 1—Preparation of Carbomer-Based Honokiol Liposome Transdermal Gel (with Carbomer as Matrix).

The gel, by weight, includes 1% magnolol, 2% carbomer 940, and water as the reminder. Carbomer was fully swollen in water of 80° C. for 12 hours, cooled to room temperature, and mixed with the prepared honokiol liposome solution under stirring for 30 minutes to obtain the carbomer-based liposome transdermal gel.

Step 2—Preparation of HPMC-Based Honokiol Liposome Transdermal Gel (with HPMC as Matrix).

The gel, by weight, includes 1% magnolol, 2% hydroxypropyl methylcellulose (HPMC), and water as the reminder. HPMC was fully dissolved in water of 80° C., cooled to room temperature, and mixed with the prepared honokiol liposome solution under stirring for 30 minutes to obtain the HPMC-based honokiol liposome transdermal gel.

Step 3—Preparation of Sodium Alginate-Based Honokiol Liposome Transdermal Gel (with Sodium Alginate as Matrix).

The gel, by weight, includes 1% honokiol as the drug content, 5% sodium alginate, and water as the reminder. Sodium alginate was dissolved in water at room temperature, degassed by ultrasound for 10 minutes, left to stand for 12 hours, and mixed with the prepared honokiol liposome solution under stirring for 30 minutes to obtain the sodium alginate-based honokiol liposome transdermal gel.

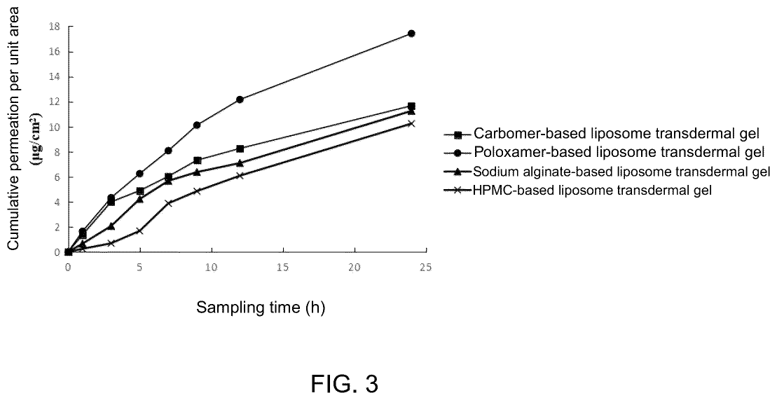

The cumulative permeation per unit area (Qn) for these gels was measured on the model skin in Example 2, and compared with the poloxamer-based liposome transdermal gel in Example 1. Results are shown in FIG. 3.

As seen in FIG. 3, the cumulative permeation per unit area (Qn) over 0-24 hours was highest for the poloxamer-based liposome transdermal gel, followed by the carbomer-based liposome transdermal gel, the sodium alginate-based liposome transdermal gel, and then the HPMC-based liposome transdermal gel. The poloxamer-based liposome transdermal gel had the highest cumulative permeation on psoriatic model skin.

### Example 4: Therapeutic Effect of Liposome Transdermal Gels with Different Matrixes on Mouse Psoriasis Model

### Step 1—Model Establishment

Sixty healthy mice were divided into six groups, with their backs shaved to expose a 2 cm×3 cm area. They were housed individually. Imiquimod cream (50 mg) was applied to their backs daily for 5 consecutive days. The mice's back skin was thickened significantly, forming plaque-like scales and some erythema, indicating successful model establishment.

### Step 2—Administration and Results

The honokiol liposome solution prepared in Example 1 (Lip group), the poloxamer-based honokiol liposome transdermal gel prepared in Example 1 (Lip-Plo-Gel group), the carbomer-based liposome transdermal gel prepared in Example 4 (Lip-CP-Gel group), the HPMC-based liposome transdermal gel (Lip-HPMC-Gel group), and the sodium alginate-based liposome transdermal gel (Lip-SA-Gel group) were used. Each was administered to the dorsal skin of mice (200 μg of magnolol) in five groups with a control group untreated. The treatment was applied twice daily for ten days, and the dorsal skin condition was observed daily. Using the PASI scoring system (0, no symptoms; 1, mild; 2, moderate; 3, severe; 4, extremely severe), the scores for erythema, skin thickness, and scaling were recorded, with the total scores on Day 10 presented in Table 1.

TABLE 1
PASI Scores on Day 10

| | | Skin | | |
|---|---|---|---|---|
| Group | Erythema | Thickness | Scaling | Total Score |
| Control | 3.0 | 3.8 | 3.5 | 10.3 |
| Lip | 1.4 | 2.7 | 2.8 | 6.9 |
| Lip-Plo-Gel | 0.6 | 1.4 | 1.8 | 3.8 |
| Lip-CP-Gel | 1.8 | 2.4 | 2.2 | 6.4 |
| Lip-HPMC-Gel | 1.2 | 2.1 | 2.5 | 5.8 |
| Lip-SA-Gel | 1.1 | 2.5 | 2.6 | 6.2 |

According to Table 1, the Lip, Lip-CP-Gel, Lip-HPMC-Gel, and Lip-SA-Gel groups all showed some therapeutic effects, while the Lip-Plo-Gel group demonstrated significant therapeutic effects, which significantly reduced erythema, skin thickness, and scaling.

### Example 5: Stability Study of Liposome Transdermal Gels with Different Matrixes

The transdermal gels of the Lip-Plo-Gel, Lip-CP-Gel, Lip-HPMC-Gel, and Lip-SA-Gel groups were respectively placed in amber vials, sealed, and stored at 40° C. with 75% humidity for 30 days. Samples were taken on Day 0 and Day 30 to compare the appearance and drug content (with Day 0 content set as 100%). The results are shown in Table 2.

TABLE 2
Appearance and Drug Content on Day 10

| | | Drug |
|---|---|---|
| Group | Appearance Change | Content |
| Lip-Plo-Gel | Semi-solid, no significant change in color | 99.6% |
| | and viscosity | |
| Lip-CP-Gel | Semi-solid, no significant change in color | 99.2% |
| | and viscosity | |
| Lip-HPMC-Gel | Semi-solid, uneven color, increased | 96.6% |
| | viscosity, layering | |
| Lip-SA-Gel | Semi-solid, slight flocculation, no | 96.3% |
| | significant change in color and viscosity | |

According to Table 2, the Lip-Plo-Gel group and the Lip-CP-Gel group maintained stable transdermal gel characteristics and consistent drug content under high-temperature and high-humidity conditions, while the Lip-HPMC-Gel group and the Lip-SA-Gel group exhibited slight layering or flocculation, and decreased drug content. Thus, the Lip-Plo-Gel group and the Lip-CP-Gel group demonstrated better stability.

### Example 6: Ratio Screening of Poloxamer and Liposome in Transdermal Gels

Using the honokiol liposome solution prepared in Example 1, with a drug content of honokiol of 1%, liposome transdermal gels with varying poloxamer 407 ratios were prepared according to Example 1. The cumulative permeation per unit area (Qn) in 24 hours was respectively measured using the skin model prepared in Example 2. The results are shown in Table 3.

TABLE 3
In Vitro Transdermal Permeation with Different
Ratios of Poloxamer and Liposome

| Honokiol | | | |
|---|---|---|---|
| Liposome:Poloxamer | Cumulative permeation | | |
| (M/M), by | per unit area Qn (μg/cm²) | | |
| Weight Ratio | 5 h | 12 h | 24 h |
| 1:1 | 5.24 | 11.48 | 15.31 |
| 1:2 | 6.35 | 12.60 | 17.97 |
| 1:4 | 6.18 | 12.14 | 17.34 |
| 1:6 | 6.57 | 12.88 | 18.46 |
| 1:8 | 5.87 | 11.62 | 16.62 |
| 1:10 | 5.33 | 11.56 | 15.12 |
| 1:12 | 5.08 | 10.15 | 12.38 |

According to Table 3, when the weight ratio of honokiol liposome to poloxamer is 1:1-1:12, the cumulative permeation per unit area (Qn) in 24 hours reaches more than 12 μg/cm²; when the weight ratio of honokiol liposome to poloxamer is 1:1-1:10, the cumulative permeation per unit area (Qn) in 24 hours reaches more than 15 μg/cm²; when the the weight ratio of honokiol liposome to poloxamer is 1:2-1:6, the cumulative permeation per unit area (Qn) in 24 hours reaches more than 17 μg/cm². Preferably, the weight ratio is 1:6, and the cumulative permeation per unit area (Qn) in 24 hours reaches more than 18 μg/cm², and the transdermal absorption effect is the best.

### Example 7

### Step 1—Preparation of Honokiol Liposome Solution

Phospholipid, honokiol (10 g), cholesterol, and polyethylene glycol-4000 in a weight ratio of 5:1:0.3:0.1 were taken and mixed uniformly, and then fully dissolved in anhydrous ethanol, then evaporated at 25° C. and 120 rpm to remove the organic solvent to obtain a uniform phospholipid film. The phospholipid film was hydrated with water for injection at 25° C., and stirred magnetically at 3000 rpm for 1.5 hours, then the product was homogenized in a high-pressure homogenizer at a homogenization pressure of 15,000 psi, and then the solution was filtered through a filter of 0.22 μm at 4° C. to obtain honokiol liposome solution.

Upon measurement, the prepared honokiol liposome had an average particle size of 128.6 nm, a PDI value of 0.233, a zeta potential of −31.7 mV, and an encapsulation efficiency of honokiol greater than 90%.

### Step 2—Preparation of Honokiol Liposome Transdermal Gel

The gel, by weight, includes 1% honokiol liposome (by film weight) prepared in Step 1, 50% poloxamer 237, and water as the reminder. The poloxamer was added to water of 3° C. and stirred for 4 hours, and fully swollen at 3° C. for 32 hours; then the prepared honokiol liposome solution was added under stirring, and stirred for 40 minutes to obtain the honokiol liposome transdermal gel.

### Example 8

### Step 1—Preparation of Honokiol Liposome Solution

Phospholipid, honokiol (10 g), cholesterol, and PEG-1000 in a weight ratio of 2:1:0.6:0.3 were taken and mixed uniformly, and then fully dissolved in anhydrous ethanol, then evaporated at 40° C. and 120 rpm to remove the organic solvent to obtain a uniform phospholipid film. The phospholipid film was hydrated with water for injection at 75° C., and stirred magnetically at 2000 rpm for 1 hour, then the product was homogenized in a high-pressure homogenizer at a homogenization pressure of 15,000 psi, and then the solution was filtered through a filter of 0.22 μm at 4° C. to obtain the honokiol liposome solution.

Upon measurement, the prepared honokiol liposome had an average particle size of 114.3 nm, a PDI of 0.257, a zeta potential of-35.8 mV, and an encapsulation efficiency of honokiol greater than 90%.

### Step 2—Preparation of Honokiol Liposome Transdermal Gel

The gel, by weight, includes 5% honokiol liposome (by film weight) prepared in Step 1, 40% poloxamer 407, 0.5% methylparaben, 10% glycerin, 0.1% triethanolamine, and water as the reminder. The poloxamer was added to water of 10° C. and stirred for 5 hours, and fully swollen at 10° C. for 24 hours. The honokiol liposome solution prepared in step 1 and other components were added under stirring, and stirred for 40 minutes to obtain the honokiol liposome transdermal gel.

### Example 9

### Step 1—Preparation of Honokiol Liposome Solution

Phospholipid, honokiol (10 g), cholesterol, and PE-PEG-2000 in a weight ratio of 6:1:0.4:0.3 were taken and mixed uniformly, and then fully dissolved in anhydrous ethanol, then evaporated at 50° C. and 120 rpm to remove the organic solvent to obtain a uniform phospholipid film. The phospholipid film was hydrated with water for injection at 60° C., and stirred magnetically at 2000 rpm for 1 hour, then the product was homogenized in a high-pressure homogenizer at a homogenization pressure of 15,000 psi, and then the solution was filtered through a filter of 0.22 μm at 4° C. to obtain the honokiol liposome solution.

Upon measurement, the prepared honokiol liposome had an average particle size of 134.6 nm, a PDI of 0.287, a zeta potential of −30.9 mV, and an encapsulation efficiency of honokiol greater than 90%.

### Step 2-Preparation of Honokiol Liposome Transdermal Gel

The gel, by weight, includes 30% honokiol liposome (by film weight) prepared in Step 1, 30% poloxamer 188, 0.2% methylparaben, 5% glycerin, 0.5% triethanolamine, 1% azone, and water as the reminder. The poloxamer was added to water of 4° C. and stirred for 1 hour, and fully swollen at 4° C. for 24 hours. The honokiol liposome solution prepared in Step 1 and other components were added under stirring, and stirred for 40 minutes to obtain the honokiol liposome transdermal gel.

### Example 10

### Step 1—Preparation of Honokiol Liposome Solution

Phospholipid, honokiol (10 g), cholesterol, and DMPE-PEG-4000 in a weight ratio of 2.5:1:0.4:0.2 were taken and mixed uniformly, and then fully dissolved in anhydrous ethanol, then evaporated at 60° C. and 120 rpm to remove the organic solvent to obtain a uniform phospholipid film. The phospholipid film was hydrated with water for injection at 40° C., and stirred magnetically at 2000 rpm for 1 hour, then the product was homogenized in a high-pressure homogenizer at a homogenization pressure of 15,000 psi, and then the solution was filtered through a filter of 0.22 μm at 4° C. to obtain the honokiol liposome solution.

Upon measurement, the prepared honokiol liposome had an average particle size of 133.2 nm, a PDI of 0.247, a zeta potential of-35.6 mV, and an encapsulation efficiency of honokiol greater than 90%.

### Step 2—Preparation of Honokiol Liposome Transdermal Gel

The gel, by weight, includes 3% honokiol liposome (by film weight) prepared in Step 1, 25% poloxamer 124, 0.1% menthol, 0.1% sodium metabisulfite, 10% propylene glycol, 2.5% diethylene glycol monoethyl ether oleate, 0.5% sodium lauryl sulfate and water as the reminder. The poloxamer was added to water of 4° C. and stirred for 1.5 hours, and fully swollen at 4° C. for 24 hours. The honokiol liposome solution prepared in Step 1 and other components were added under stirring, and stirred for 40 minutes to obtain the honokiol liposome transdermal gel.

### Example 11

### Step 1—Preparation of Honokiol Liposome Solution

Phospholipid, honokiol (10 g), cholesterol, and PC-PEG-2000 in a weight ratio of 3.5:1:0.5:0.2 were mixed uniformly, and then chloroform/ethanol (1:5 v/v) was added to fully dissolve the mixture, then evaporated at 45° C. and 120 rpm to remove the organic solvent to obtain a uniform phospholipid film. The phospholipid film was hydrated with water for injection at 50° C., and stirred magnetically at 2000 rpm for 1 hour, then the product was homogenized in a high-pressure homogenizer at a homogenization pressure of 15,000 psi, and then the solution was filtered through a filter of 0.22 μm at 4° C. to obtain the honokiol liposome solution.

Upon measurement, the prepared honokiol liposome had an average particle size of 116.9 nm, a PDI of 0.256, a zeta potential of-31.1 mV, and an encapsulation efficiency of honokiol greater than 90%.

### Step 2—Preparation of Honokiol Liposome Transdermal Gel

The gel, by weight, includes 8% honokiol liposome (by film weight) prepared in Step 1, 22% poloxamer 181, 5% stearic acid, 0.2% Tween 80, and water as the reminder. The poloxamer was added to water of 6° C. and stirred for 2 hours, and fully swollen at 6° C. for 72 hours. The honokiol liposome solution prepared in Step 1 and other components were added under stirring, and stirred for 25 minutes to obtain the honokiol liposome transdermal gel.

### Example 12

### Step 1—Preparation of Honokiol Liposome Solution

Phospholipid, honokiol (10 g), cholesterol, and DSPE-PEG-2000 in a weight ratio of 4.5:1:0.4:0.2 were mixed uniformly. Anhydrous ethanol was added, and the mixture was slowly and uniformly injected into deionized water under ultrasound conditions at 25° C. The ethanol was removed by rotary evaporation under reduced pressure to obtain the honokiol liposome solution.

Upon measurement, the prepared honokiol liposome had an average particle size of 106.2 nm, a PDI value of 0.368, a zeta potential of −38.4 mV, and an encapsulation efficiency of honokiol greater than 90%.

### Step 2—Preparation of Honokiol Liposome Transdermal Gel

The gel, by weight, includes 16% honokiol liposome (by film weight) prepared in Step 1, 45% poloxamer 331, and water as the reminder. The poloxamer was added to 4° C. water and stirred for 2 hours, then fully swollen at 4° C. for 32 hours. The honokiol liposome solution prepared in Step 1 was added under stirring, and stirred for 35 minutes to obtain the honokiol liposome transdermal gel.

### Example 13

### Step 1—Preparation of Honokiol Liposome Solution

Phospholipid, honokiol (10 g), cholesterol, and DPPC-PEG-2000 in a weight ratio of 3:1:0.4:0.1 were mixed uniformly. Anhydrous ethanol was added, and the mixture was slowly and uniformly injected into deionized water under ultrasound conditions at 25° C. The ethanol was removed by rotary evaporation under reduced pressure to obtain the honokiol liposome solution.

Upon measurement, the prepared honokiol liposome had an average particle size of 117.8 nm, a PDI value of 0.294, a zeta potential of −30.9 mV, and an encapsulation efficiency of honokiol greater than 90%.

### Step 2—Preparation of Honokiol Liposome Transdermal Gel

The gel, by weight, includes 30% of the honokiol liposome (by film weight) prepared in Step 1, 30% poloxamer 182, and water as the reminder. The poloxamer was added to 4° C. water and stirred for 2 hours, then allowed to fully swell at 4° C. for 26 hours. The honokiol liposome solution prepared in Step 1 was added under stirring, and stirred for 35 minutes to obtain the honokiol liposome transdermal gel.

Transdermal penetration experiments were conducted on the transdermal gels prepared in Examples 7-13 according to the penetration experiment on model skin of Example 2, for measuring the cumulative permeation per unit area (Qn) over 0-24 hours. The results showed that the cumulative permeation per unit area (Qn) at 24th hour was 11.39 to 17.28 μg/cm², indicating that the honokiol liposome transdermal gels prepared in the present invention had good transdermal penetration effects.

What is claimed is:

1. A honokiol liposome transdermal gel, comprising by weight percentage:
1-40% honokiol liposome and 10-85% gel matrix;
wherein the honokiol liposome comprises phospholipid, honokiol, cholesterol, and polyethylene glycol or pegylated phospholipid, in a weight ratio of 2-10:1:0.3-0.6:0.1-0.3; and
the gel matrix is poloxamer.

2. The honokiol liposome transdermal gel according to claim 1, wherein a weight ratio of the honokiol liposome to the poloxamer is 1:1-1:10.

3. The honokiol liposome transdermal gel according to claim 1, wherein a weight ratio of the honokiol liposome to the poloxamer is 1:2-1:6.

4. The honokiol liposome transdermal gel according to claim 1, wherein the poloxamer is selected from any one or a combination of poloxamer 407, poloxamer 188, poloxamer 338, poloxamer 237, poloxamer 124, poloxamer 181, poloxamer 182, and poloxamer 331.

5. The honokiol liposome transdermal gel according to claim 1, wherein the poloxamer is poloxamer 407 and/or poloxamer 188.

6. The honokiol liposome transdermal gel according to claim 1, further comprising an additive selected from any one or a combination of antioxidants, transdermal enhancers, moisturizers, pH adjusters, preservatives, and thickeners.

7. A preparation method of the honokiol liposome transdermal gel according to claim 1, comprising the following steps:
(1) preparation of honokiol liposome: taking and uniformly mixing phospholipid, cholesterol, polyethylene glycol or pegylated phospholipid, and honokiol, adding organic solvent to fully dissolve, evaporating to remove the organic solvent to obtain a uniform phospholipid film, adding water to the phospholipid film, stirring for 1-1.5 h, homogenizing to obtain a liposome solution; filtering the liposome solution at 2-10° C. with a filter of 0.22 μm to obtain the honokiol liposome;
(2) preparation of honokiol liposome transdermal gel: stirring poloxamer with water for 0.5-5 h until fully swollen, then adding the honokiol liposome obtained in step (1) and an additive during stirring, and stirring for 20-40 minutes to obtain the honokiol liposome transdermal gel.

8. The preparation method according to claim 7, wherein the organic solvent in step (1) is selected from any one or a combination of anhydrous ethanol, chloroform, methylene chloride, and methanol.

9. The preparation method according to claim 8, wherein the poloxamer in step (2) is stirred with water of 2-10° C., with a swelling temperature of 2-10° C., and a swelling time of 24-72 h.

10. The preparation method according to claim 7, wherein a weight ratio of the honokiol liposome to the poloxamer is 1:1-1:10.

11. The preparation method according to claim 7, wherein a weight ratio of the honokiol liposome to the poloxamer is 1:2-1:6.

12. The preparation method according to claim 7, wherein the poloxamer is selected from any one or a combination of poloxamer 407, poloxamer 188, poloxamer 338, poloxamer 237, poloxamer 124, poloxamer 181, poloxamer 182, and poloxamer 331.

13. The preparation method according to claim 7, wherein the poloxamer is poloxamer 407 and/or poloxamer 188.

14. The preparation method according to claim 7, wherein the additive is selected from any one or a combination of antioxidants, transdermal enhancers, moisturizers, pH adjusters, preservatives, and thickeners.

15. A use of the honokiol liposome transdermal gel as claimed in claim 1, comprising applying the honokiol liposome transdermal gel in a preparation of a formulation for preventing and/or treating psoriasis.

16. The use of honokiol liposome transdermal gel according to claim 15, wherein a weight ratio of the honokiol liposome to the poloxamer is 1:1-1:10.

17. The use of honokiol liposome transdermal gel according to claim 15, wherein a weight ratio of the honokiol liposome to the poloxamer is 1:2-1:6.

18. The use of honokiol liposome transdermal gel according to claim 15, wherein the poloxamer is selected from any one or a combination of poloxamer 407, poloxamer 188, poloxamer 338, poloxamer 237, poloxamer 124, poloxamer 181, poloxamer 182, and poloxamer 331.

19. The use of honokiol liposome transdermal gel according to claim 15, wherein the poloxamer is poloxamer 407 and/or poloxamer 188.

20. The use of honokiol liposome transdermal gel according to claim 15, wherein the honokiol liposome transdermal gel further comprises an additive selected from any one or a combination of antioxidants, transdermal enhancers, moisturizers, pH adjusters, preservatives, and thickeners.
